(19) United States
(12) Patent Application Publication
(10) Pub. No.: US 2026/0171187 A1
(43) Pub. Date: Jun. 18, 2026

(54) SYSTEMS AND METHODS FOR USING NATURAL LANGUAGE PROCESSING (NLP) TO PREDICT PROTEIN FUNCTION SIMILARITY

(71) Applicant: BASE SE, Ludwigshafen am Rhein (DE)

(72) Inventors: Jongmin BAEK, Cary, NC (US); Sebastian Hermann MARTSCHAT, Ludwigshafen am Rhein (DE); Soheila SAMIEE, Mississauga (CA)

(21) Appl. No.: 19/127,056

(22) PCT Filed: Oct. 30, 2023
(86) PCT No.: PCT/IB2023/060914

§ 371 Date: May 2, 2025

Related U.S. Application Data
(60) Provisional application No. 63/421,857, filed on Nov. 2, 2022.

(51) Int. Cl. G16B 15/00; G16B 40/00; G16B 50/30

(52) U.S. Cl. CPC G16B 15/00; G16B 40/00; G16B 50/30

(57) ABSTRACT

Systems and methods are provided that may analyze protein sequences using a natural language processing (NLP) model to, for example, detect structurally similar proteins in a database of unclassified proteins. Systems and methods are also provided to apply a secondary model to tune the NLP model during a training phase. Systems and methods are also provided to train the secondary model using a database of hierarchical structural classifications. As such, the NLP model is tuned to output embedding vectors that indicate structural characteristics for a protein corresponding to an input protein sequence.

### TECHNICAL FIELD

The present disclosure generally relates to systems and methods to predict structural similarity between proteins. More particularly, the present disclosure relates to identification of proteins having similar structural features to a target protein using natural language processing (NLP).

### BACKGROUND

Proteins are formed by one or more chains of amino acids. By following a standardized process, these chains can be broken down into a sequence of amino acid residues that represents the genetic makeup of the protein. This sequence of amino acid residues is also referred to as a “protein sequence.” Accordingly, a protein can be represented by its protein sequence.

Generally, the protein sequence encodes the structure of a protein. Because the structure of a protein is often correlated to the function of a protein, the protein sequence is also generally understood to encode the protein function. However, the structure of most proteins is not known. As a result, if a target protein has been determined to have a particularly beneficial trait (e.g., insecticidal properties), it is useful to identify similarities in the protein sequence to identify candidate proteins having an unknown structure that may exhibit the same trait. This enables protein engineers to cast a wider net in investigating which protein(s) are best suited to perform a specific function.

Past attempts to identify similarity between proteins focused on protein sequence alignment. These techniques attempt to identify particular portions of the protein sequence that are believed to be particularly consequential and perform a similarity analysis of these sequences. However, proteins can exhibit the same function even if the specific sequences are not similar. Thus, an alignment analysis is limited in its ability to detect candidate proteins that exhibit a desired function. Additionally, the alignment analysis is computationally expensive to perform. As a result, searching a database of millions of protein sequences to identify candidate proteins using an alignment analysis also takes a significant amount of time.

Accordingly, to overcome the drawbacks of alignment analyses, others have turned to natural language processing (NLP) techniques to analyze the protein sequences. One such model of how protein sequences are similar to natural language grammar is described in Gimona, Mario, Protein Linguistics—a Grammar for Modular Protein Assembly, Nature Reviews Molecular Cell Biology, Vol. 7(2006 ). Rather than simply comparing the raw protein sequences, the NLP approaches attempt to apply deeper learning to better understand the relationship the protein sequence and protein function in a comparable way the NLP models are more commonly applied to derive similarity between words or concepts.

However, naively applying NLP techniques to a protein sequence database and identifying similarity between the resulting feature vectors does not account for the structural similarity that is particular to the protein sequence context. As a result, while the naive NLP techniques are able to predict a subsequent amino acid in a sequence of amino acids, the naïve NLP techniques actually fared worse than the alignment approach when identifying structural similarity. This was expected because the naïve NLP model is not trained with the structural similarity goal in mind.

One approach to improving the naive NLP techniques to protein sequences is described in Bepler, Tristan, and Berger, Bonnie, Learning Protein Sequence Embeddings Using Information from Structure, International Conference on Learning Representations (2019), available at https://arxiv.org/abs/1902.08661 (“Bepler and Berger”). FIGS. 1A and 1B depict representations of the Bepler and Berger approach. In particular, FIG. 1A depicts the training phase in the Bepler and Berger approach and FIG. 1B depicts the inference phase in the Bepler and Berger approach.

Generally, in the Bepler and Berger approach, a recurrent neural network (RNN) 12 is applied on top of a pre-trained NLP neural network 10 (in particular, a bidirectional LSTM model) that accepts a protein sequence 5 as an input. The RNN 12 is trained to output a plurality of feature vectors 15 that are combined into a vector embedding 20. Because protein sequences 5 can have variable lengths, the vector embeddings 20 can have different lengths. Accordingly, the Bepler and Berger approach includes a soft symmetric alignment (SSA) layer 22 that normalizes the length of the vector embeddings 20 such that a similarity calculation technique can be performed.

During the training phase, the Bepler and Berger approach trains the RNN 12. More particularly, as illustrated, the Bepler and Berger approach applies a top level model 25 to train the RNN 12 while leaving the NLP neural network 10 as is. The top level model 25 is trained based upon a number of levels shared by two known proteins in a hierarchical database of protein structures, such as the Structural Classification of Proteins (SCOP) database.

However, the Bepler and Berger approach has several drawbacks. As mentioned above, the vector embeddings 20 have different lengths depending on the particular protein sequence 5 input into NLP neural network 10. Accordingly, as illustrated in FIG. 1B, the SSA layer 22 is also present in the inference phase. The SSA layer 22 is a processor-intensive calculation that significantly increases the amount of time required to search an index of protein structures to identify proteins that are predicted to have a similar structure. As such, the Bepler and Berger approach is not scalable to large data sets. Additionally, the Bepler and Berger approach requires applying two different neural networks to generate the feature vectors 15. Further, the Bepler and Berger approach only trains the RNN 12, not the underlying NLP neural network 10. Accordingly, the Bepler and Berger approach does not fully leverage the deep learning capabilities of the NLP neural network.

In view of the foregoing challenges, there is a need for improved systems and methods for applying NLP techniques to predict functional similarity between proteins.

### SUMMARY

In an embodiment, a system for predicting functional similarity between proteins is provided. The system comprises (i) one or more processors; (ii) a first one or more non-transitory memories configured to store a primary machine learning model configured to convert a protein sequence for a protein into an embedding vector representative of features of the protein, wherein the primary machine learning model is trained or fine-tuned by (a) training a secondary machine learning model to predict a structural similarity between two proteins, (b) inputting a plurality of protein sequence pairs into the primary machine learning model to obtain pairs of embedding vectors, (c) inputting the obtained pairs of embedding vectors into the secondary machine learning model to obtain a respective predicted structural similarity between proteins represented by the pairs of protein sequences, and (d) tuning the primary machine learning model based upon the predicted structural similarities, (iii) a second one or more non-transitory memories configured to store processor-executable instructions. The instructions, when executed by the one or more processors, cause the system to (1) receive an indication of an input protein sequence; (2) generate, using the primary machine learning model, an embedding vector for the input protein sequence; (3) compare the embedding vector for the input protein sequence to a plurality of embedding vectors for a plurality of candidate protein sequences by applying a similarity operation; and (4) ranking candidate protein sequences based upon outputs of the similarity operation.

In another embodiment a computer-implemented method for predicting functional similarity between proteins using a primary machine learning model is provided. The primary machine learning model is configured to convert a protein sequence for a protein into an embedding vector representative of features of the protein and trained by (i) training a secondary machine learning model to predict a structural similarity between two proteins, (ii) inputting a plurality of protein sequence pairs into the primary machine learning model to obtain pairs of embedding vectors, (iii) inputting the obtained pairs of embedding vectors into the secondary machine learning model to obtain a respective predicted structural similarity between proteins represented by the pairs of protein sequences, and (iv) tuning the primary machine learning model based upon the predicted structural similarities. The method includes (1) receiving, via one or more processors, an indication of an input protein sequence; (2) generating, using the primary machine learning model, an embedding vector for the input protein sequence; (3) comparing, via the one or more processors, the embedding vector for the input protein sequence to a plurality of embedding vectors for a plurality of candidate protein sequences by applying a similarity operation; and (4) ranking, via the one or more processors, candidate protein sequences based upon outputs of the similarity operation.

In a further embodiment, a computer-implemented method of training or fine-tuning a model to predict functional similarity between proteins is provided. The method includes (1) training, via one or more processors, a secondary machine learning model to predict a structural similarity between two proteins using a database of hierarchical structural classifications for a plurality of proteins; (2) inputting, via the one or more processors, a plurality of protein sequence pairs into a pre-trained natural language processing (NLP) model to obtain pairs of embedding vectors; (3) inputting, via the one or more processors, the obtained pairs of embedding vectors into the secondary machine learning model to obtain a respective predicted structural similarity between proteins represented by the pairs of protein sequences; and (4) tuning, via the one or more processors, the NLP model based upon the predicted structural similarities.

### BRIEF DESCRIPTION OF THE FIGURES

The Figures described below depict various aspects of computer-implemented methods, systems comprising computer-readable media, and electronic devices disclosed therein. It should be understood that each Figure depicts an embodiment of a particular aspect of the disclosed methods, media, and devices, and that each of the figures is intended to accord with a possible embodiment thereof. Further, wherever possible, the following description refers to the reference numerals included in the following Figures, in which features depicted in multiple Figures are designated with consistent reference numerals. The present embodiments are not limited to the precise arrangements and instrumentalities shown in the Figures.

FIGS. 1A and 1B depict the training phase and inference phase for a prior machine learning model architecture that applied NLP processing to protein sequencing;

FIGS. 2A and 2B depict the training phase and inference phase for the instant machine learning model architecture that applies NLP processing to protein sequencing in an improved manner;

FIG. 3 depicts an example computing environment in which the disclosed protein function prediction techniques are implemented.

FIG. 4A depicts an example user interface for inputting a protein sequence into a search interface.

FIG. 4B depicts an example user interface for presenting search results.

FIG. 5 depicts an example flow diagram of a method for training a model to predict functional similarity between proteins.

FIG. 6 depicts an example flow diagram of a method for predicting functional similarity between proteins.

The Figures depict aspects of the present disclosure for purposes of illustration only. One skilled in the art will readily recognize from the following discussion that alternate aspects of the structures and methods illustrated herein may be employed without departing from the principles of the disclosure described herein.

### DETAILED DESCRIPTION

Systems and methods are provided for improved techniques for applying natural language processing (NLP) to protein sequences to predict functional similarity. More specifically, the systems and methods of the present disclosure may implement NLP to identify at least one candidate protein that is predicted to have a similar function to an input protein.

The system, and methods of the present disclosure may improve upon prior efforts to apply NLP to protein sequences by, for example, reducing the amount of time that it takes to perform a search of a protein sequence database. In our experimental testing on a database that has 20 million protein sequences, the instant techniques were able to conduct a search of the database in ˜20 seconds, whereas the Bepler and Berger approach described above required ˜3 hours. As will be described below, the reduced time to provide search results enables searching software to provide additional searching capabilities that would otherwise take an impractical amount of time.

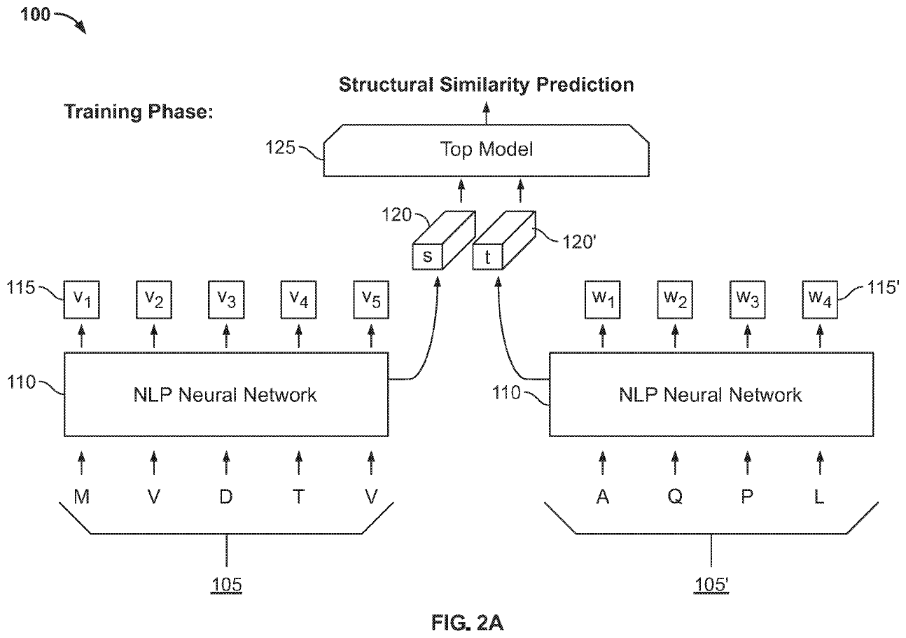

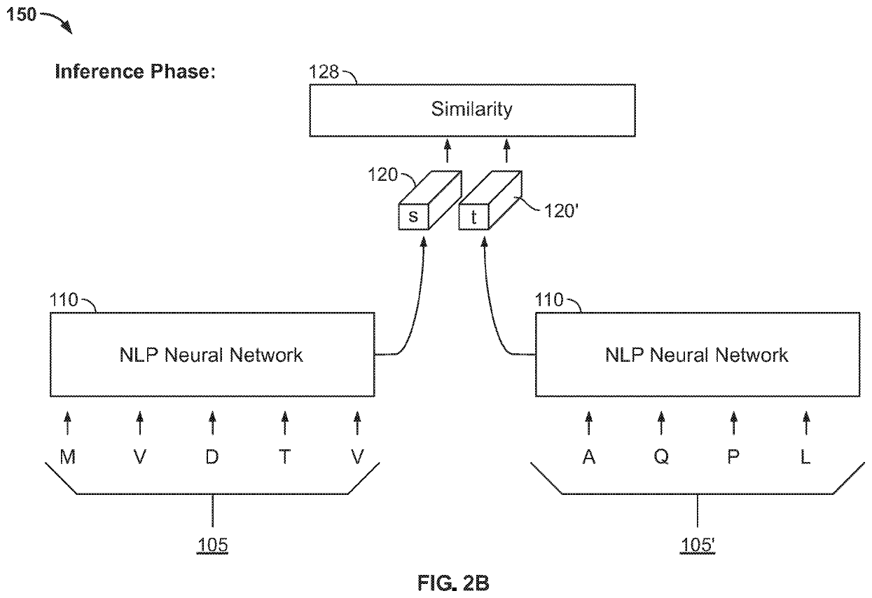

With reference to FIGS. 2A and 2B, depicted is a machine learning model architecture configured in accordance with the instant techniques is depicted. More particularly, FIG. 2A depicts the machine learning model architecture 100 during the training phase and FIG. 2B depicts the machine learning model architecture 150 during the inference phase (e.g., when the machine learning model is applied to perform a search of a protein sequence database). This approach is inspired by Reimers, N., & Gurevych, I. [2019]. (Sentence-BERT: Sentence Embeddings using Siamese BERT-Networks, available at https://arxiv.org/pdf/1908.10084.pdf) which applies the disclosed techniques to the NLP field.

Starting with the training phase depicted in FIG. 2A, the machine learning model architecture 100 includes a NLP neural network 110 configured to accept a protein sequence 105 as an input and output a series of vectors vₙ 115 that represent the individual amino acid residues and an embedding vector s 120 that represents a plurality of features of the input protein sequence 105.

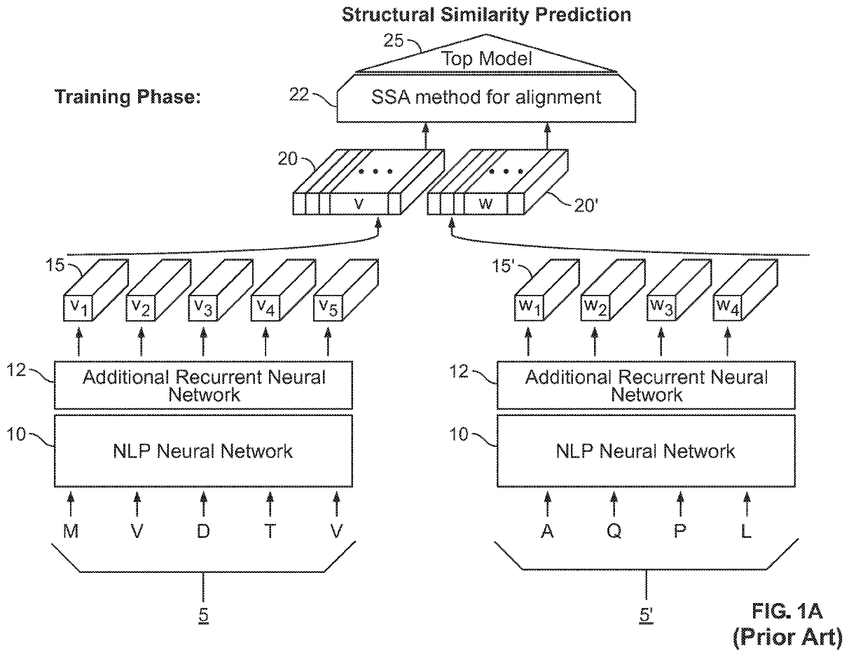

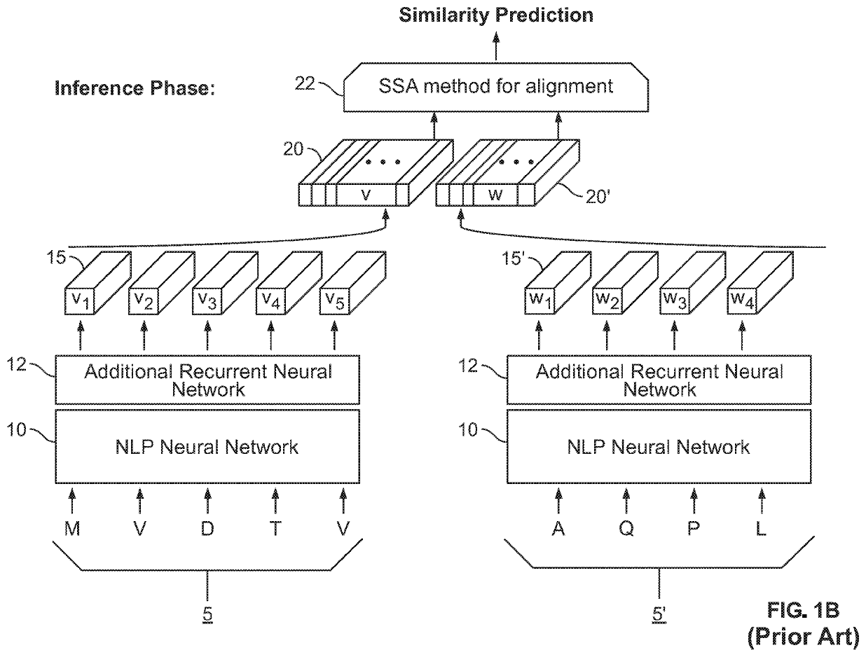

In some embodiments, the NLP neural network 110 is pre-trained using a large database of proteins. For example, the NLP neural network 110 may be a bidirectional encoder representations from transformers (BERT) model adapted for protein sequencing (e.g., ProtBERT), an evolutionary scale modeling (ESM) model, or another open pre-trained NLP model. Typically, the pre-trained NLP models include a normalizer layer that normalizes the output embedding vector to a common length. For example, the normalizer layer may average the embedding vectors or using the first token (e.g., the [CLS] token). FIG. 1A of Bepler and Berger suggests a SSA approach to achieve a better representation compared to averaging; however, as mentioned above, it would lead to higher computation cost, and slower inference.

In some embodiments, if the NLP neural network 110 model does not include a normalizer layer or if the normalizer is not optimized for the task, the NLP neural network 110 may be modified (i.e. fine-tuned) to provide a proper normalized output. Hence, by selecting a pre-trained NLP model that includes a normalizer (or modifying a NLP model), one may achieve the same goal without applying sequence alignment techniques.

During the training process, the NLP neural network 110 is fine-tuned based upon a predicted structural similarity between two embedding vectors 120, 120′ for the protein sequences 105, 105′. For example, the NLP neural network 110 may be fine-tuned using target-oriented approaches that use the predicted structural similarity as the target. By fine-tuning the NLP neural network 110 in this manner, the features captured in the embedding vectors 120 output by the NLP neural network 110 are tuned to be indicative of protein structure such that a structural similarity (and hence functional similarity) can be predicted.

To predict the similarity between the embedding vectors 120, 120′, the machine learning model architecture 100 includes a top model 125. The top model may be a neural network, such as a recurrent neural network (RNN), trained on a set of pre-classified protein sequences. For example, one type of database of pre-classified protein sequences are the Structural Classification of Proteins (SCOP) and Structural Classification of Proteins-extended (SCOPe) databases. Another example database type is the Pfam databases maintained by Xfam. Additionally, protein engineering companies may develop their own databases that apply the SCOP hierarchy to add the proteins that are not included in the SCOP or SCOPe databases. The SCOP hierarchy breaks down a protein into several different hierarchical layers (e.g., class, fold, superfamily, family, protein, species, etc.) indicating the structure of the corresponding protein. It should be appreciated that other types of hierarchies may also be applied.

Accordingly, a structural similarity metric may be defined to indicate the closeness of relation between the protein sequences 105, 105′ in the SCOP (or other) hierarchy. For example, the structural similarity metric may indicate a number of layers shared by the proteins 105, 105′. As another example, the structural similarity metric may assign a higher weight to lower levels in the structural hierarchy that are more likely to result in functional similarity. In some embodiments, the machine learning model architecture 100 may modify the structural similarity metric based upon a sequence similarity. That is, the machine learning model architecture may assign a higher structural similarity score to protein pairs that share hierarchical layers but have low sequence similarity. Often, such pairs have similar functions.

Regardless of the particular structural similarity metric, by selecting protein sequences 105, 105′ from these databases of pre-classified proteins, the top model 125 may be trained using the known values for the structural similarity metric for the selected protein sequences 105, 105′. As a result, the top level model 125 may be able to accurately predict the structural similarity metric value for two protein sequences 105, 105′ even if one or both of the protein sequences are not pre-classified by a database.

During the training phase, the NLP neural network 110 may be fine-tuned until a training metric threshold is achieved. The training metric may be an accuracy metric, a validation loss metric, a training loss matric, and/or a combination thereof. During the training phase a subset of the protein sequences in the pre-classified databases may be designated as a validation set for the NLP neural network 110. To avoid biasing the NLP neural network 110, the training process does not select protein sequences from within the validation set while performing the fine-tuning process. In some embodiments, the training metric is re-calculated after each epoch. If the NLP neural network 110 achieves the training metric threshold, then the NLP neural network 110 may be considered sufficiently tuned and the NLP neural network 110 is ready to be applied in an inference phase.

FIG. 2B depicts the machine learning model architecture 150 during the inference phase. As illustrated, the machine learning model architecture 150 does not include the top model 125. This is because the NLP neural network 110 was fine-tuned during the training phase to output embedding vectors 120 that indicate structural characteristics of the input protein sequence. Said another way, the NLP neural network 110 is sufficiently tuned such that the output embedding vectors 120 reflect the set of features that indicate the structure for the protein in a standardized manner. As such, the embedding vectors 120, 120′ can be compared directly via a matrix similarity operation 128. For example, the matrix similarity operation 128 may be a dot product, a cosine similarity, a Euclidean similarity, and/or other matrix similarity operation. As a result, the machine learning model architecture 150 is able to quickly predict a structural similarity between the protein sequences 105, 105′ thereby identifying candidate proteins that are likely to share a similar function.

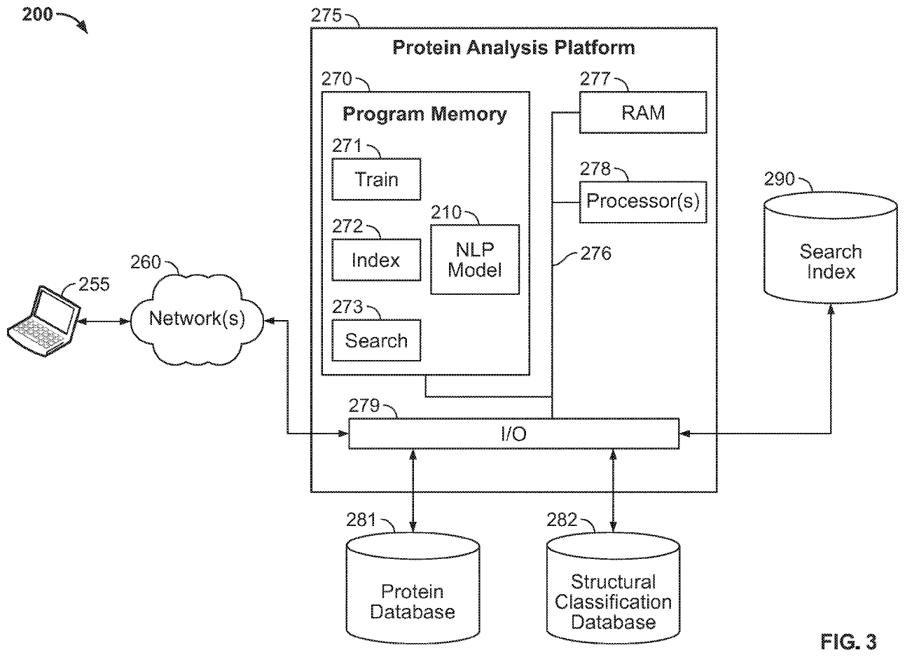

Turning to FIG. 3, depicted is an example computing environment 200 in which the disclosed protein function prediction techniques are implemented. More particularly, the environment 200 includes a protein analysis platform 275 configured to implement the disclosed protein function prediction techniques. The protein analysis platform 275 includes one or more processors 278 configured to execute instructions that form the various applications, modules, and other components of the protein analysis platform 275 described herein. The processors 278 may include central processing units (CPUs), graphics processing units (GPUs), application-specific integrated circuits (ASICS), and/or any other types of computer processors. While the disclosure may generally refer to the processors 278 executing the various tasks described herein, particular tasks may be better suited to one type of processor. For example, the repetitive analysis associated with some forms of machine learning may be more efficiently executed by GPUs than CPUs. Accordingly, in embodiments that include multiple types of processors, the protein analysis platform 275 may utilize a particular type of processor to execute instructions that is more efficiently executed by the particular type of processor.

Additionally, it should be appreciated that while FIG. 3 illustrates the protein analysis platform 275 as a single block, the protein analysis platform 275 may be multiple entities acting in conjunction with one another. For example, in some embodiments, the protein analysis platform 275 is implemented as service hosted by a distributed computing environment, such as a cloud computing environment. In these embodiments, the processors 278 may be physically located in different hardware entities (e.g., servers) despite the processors 278 being logically connected to execute the various tasks described herein.

A user interfaces with the protein analysis platform 275 via a personal electronic device 255, such as a mobile phone, a laptop computer, a tablet, a smart wearable device (e.g., smart glasses, a smart watch), a home personal assistance device, or any other electronic device that is normally used to access internet-based content. The personal electronic device 255 is communicatively coupled to the protein analysis platform 275 via one or more wired or wireless networks 260 that facilitate any type of data communication via any current or future-developed standard or technology (e.g., GSM, CDMA, TDMA, WCDMA, LTE, EDGE, OFDM, GPRS, EV-DO, UWB, IEEE 802 including Ethernet and Wi-Fi, WiMAX, Bluetooth, and others). Although FIG. 3 only illustrates one personal electronic device 255, the environment 200 may include any number of personal electronic device 255.

In the illustrated embodiment, the protein analysis platform 275 also includes a program memory 270, a random-access memory (RAM) 277, and an input/output (I/O) module 279, all of which may be interconnected via an address/data bus 276. It should be appreciated the memory of the protein analysis platform 275 may include multiple RAMs 277 and multiple program memories 270 implemented as any type of memory, such as semiconductor memory, magnetically readable memory, or optically readable memory, for example. Similarly, although the I/O module 279 is shown as a single block, it should be appreciated that the I/O module 279 may include a number of different types of I/O modules. For example, the I/O module 279 may include one or more transceiver circuits to facilitate communications over the networks 260 and/or other interconnected systems and/or databases.

The program memory 270 may store any number of applications, routines, models, or other collections of computer-readable instructions that support the protein function prediction techniques described herein. For example, the program memory 270 may include a training application 271 configured to train a NLP model 210 (such as the NLP neural network 110 of FIGS. 2A-2B), an indexing application 272 configured to generate and/or re-index a search index 290, and a search application 273 configured to conduct a search of the search index 290 to detect proteins that are predicted to have structural similarity (and hence a possible functional similarity) to an input protein. It should be appreciated that not all of the applications in the program memory 270 are accessible by all personal electronic devices 255. For example, the training application 271 and indexing application 272 may only be accessible to personal electronic devices 255 associated with a service provider for the protein analysis platform 275.

Starting with the training application 271, the training application 271 may be configured to implement the training techniques described with respect to the training phase depicted by FIG. 2A. When the training application 271 is first executed, the training application 271 may load the pre-trained NLP model 210 into the program memory 270. Additionally, the training application 271 may load a top model (not depicted) into the program memory 270. As described with respect to FIG. 2A, the training application 271 may be configured to train the top model based on pre-defined hierarchical classifications of protein structures stored in a database 282 (such as the SCOP, SCOPe, Pfam, and/or a proprietary database). The training application 271 may be configured to fine-tune the NLP model 210 until a threshold training metric is detected. In response, the training application 271 may be configured to invoke the indexing application 272 to generate the search index 290.

In particular, the indexing application 272 may be configured to generate the search index for proteins sequences stored in a protein database 281. In some embodiments, the protein database 281 includes an open database of known protein sequences, such as a UniProt Reference Clusters database (UniRef) (e.g., the UniRef100 database). Accordingly, the indexing application 272 may be configured to input the protein sequences for proteins in the protein database 281 into the NLP model 210 to generate respective embedding vectors for the proteins. The indexing application 272 may store the protein sequences and the corresponding embedding vectors in the search index 290. It should be appreciated that in most embodiments only a relatively small portion of the proteins in the protein database 281 are pre-classified in the structural classification database 282. As such, building the search index 290 enables the discovery of functionally similar proteins by detecting proteins predicted to be structurally similar to an input protein sequence.

The search application 273 may be configured to enable users to conduct a search of the search index using an input protein sequence. For example, the user may input a protein sequence for a protein that exhibits a function (e.g., insecticidal tendency) to identify other candidate proteins that are likely to exhibit the same function. This enables protein engineers to broaden their investigations when deciding which protein is best suited to perform the desired function in a given product. In some embodiments, the search application 273 is configured to accept the input protein sequence as a string of text, as a fasta file, and/or via a selection interface configured to present indications of the proteins maintained in the protein database 281. In response, the search application 273 inputs the input protein sequence into the fine-tuned NLP model 210 to generate an embedding vector for the input protein sequence. The search application 273 may then perform a similarity operation (e.g., dot product, cosine similarity, Euclidean similarity) using the embedding vector for the input protein sequence and the protein sequences in the search index 290. The search application 273 may then rank the protein sequences in the search index 290 based upon the similarity operation and present the results to the user.

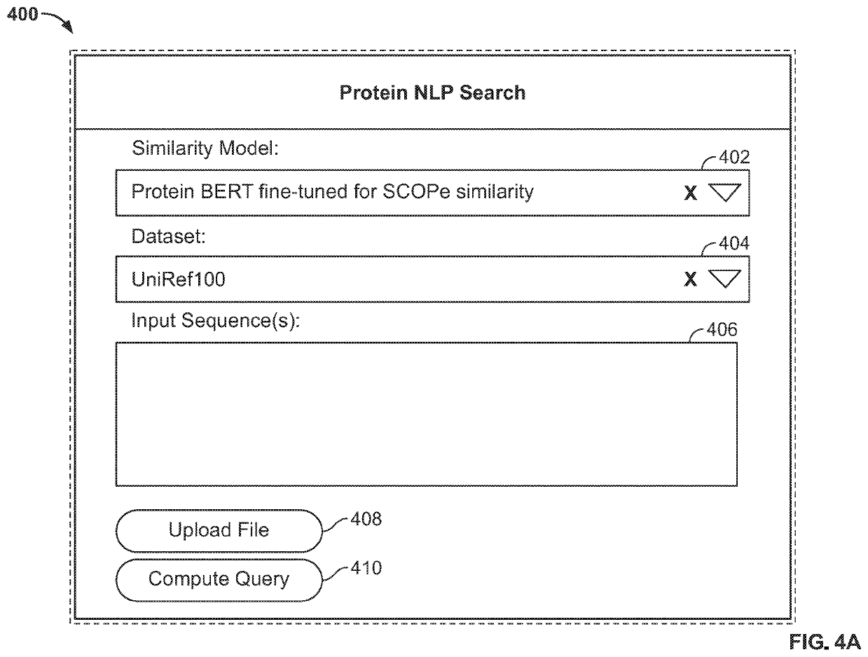

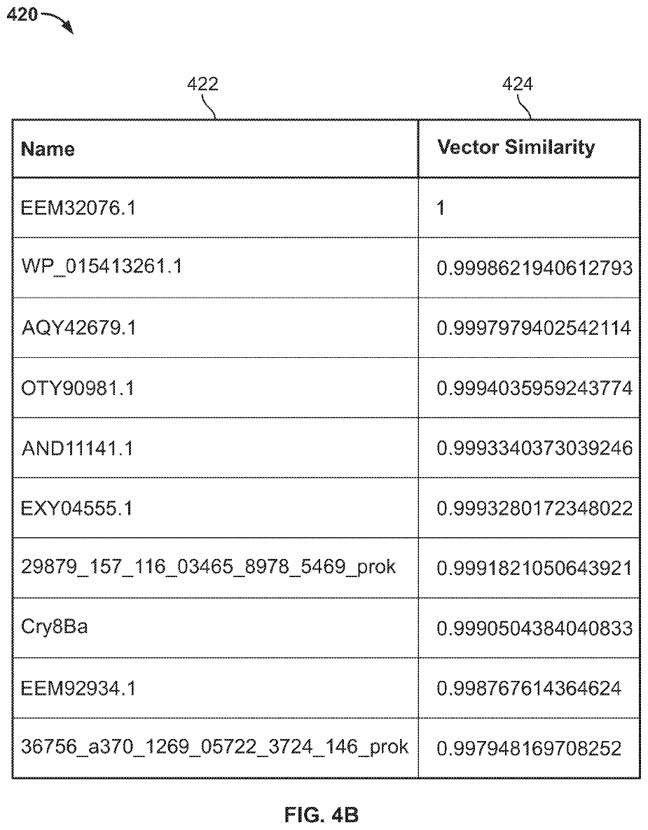

FIG. 4A depicts a user interface 400 for performing a search of a search index, such as the search index 290 to detect structurally similar proteins based upon an input protein sequence. The user interface 400 may be displayed by a personal electronic device, such as the personal electronic device 255, interfacing with a search application, such as the search application 273, hosted by a protein analysis platform, such the protein analysis platform 275.

As illustrated the user interface 400 includes a selection element 402 that enables the user to select a particular similarity model for predicting the structural similarity. To this end, in some embodiments, the protein analysis platform may be configured to train and store multiple NLP models. For example, the protein analysis platform may include a first NLP model that is a fine-tuned ProtBERT model and a second NLP model that is a fine-tuned ESM model. In addition to having different pre-trained models as a starting point, the protein analysis platform may be configured to be fine-tuned using a different hierarchical classification database. Accordingly, the protein analysis platform may include a first ProtBERT model fine-tuned using SCOPe data and a second ProtBERT model fine-tuned using a propriety data set. To this end, if a protein engineering company is interested in detecting a particular type of structures, the protein engineering company may generate a training set of protein sequences that exhibit the structures of concern.

It should be appreciated that in embodiments where the protein analysis platform maintains multiple NLP models, when an indexing application, such as the indexing application 272, generates the search index, the indexing application may create a data structure for each protein sequence that includes the embedding vector output by each NLP model. If the protein analysis platform is updated to support additional NLP models, then the data structure may be expanded to include the embedding vectors for the additional models. As a result, the user interface 400 is able to provide the ability to use the selection element 402 to select between multiple similarity models without needing to generate a new search index before performing the search.

The user interface 400 also includes a selection element 404 that enable the user to select a dataset within the search index. To this end, the data structure associated with the protein sequences in the search index may include a field that includes a list of datasets to which the protein sequence belongs. For example, the protein sequences may be labeled as belonging to the UniRef100 dataset, a UniRef50 dataset, a custom dataset of proteins currently under investigation, or other datasets. Accordingly, the selection element 404 may be populated based upon the particular labels assigned to protein sequences within the search index 290.

Additionally, the user interface includes a text entry field 406 via which the user inputs one or more input protein sequences. While the user can manually protein sequences into the text entry field 406, the protein sequences are often of such a length that manual entry is prone to error. Accordingly, the user interface 400 also includes an element 408 the enables the user to upload a file (e.g., a txt file or a fasta file) to the protein analysis platform such that protein sequences indicated in the file are automatically input into the text entry field 406. The user can then user typical text entry techniques to modify and/or remove the input protein sequences.

The user interface 400 also includes an element 410 that enables the user to initiate a search. In response to detecting an interaction with the element 410, search application may input any input protein sequences in the text entry field 406 into the NLP model indicated via the selection element 402 to generate input embedding vectors that respectively correspond to the input protein sequences. The search application then performs a similarity operation using the input embedding vectors and the embedding vectors stored in the search index and matching the dataset indicated via the selection element 404. The search application then sorts and/or ranks the protein sequences in the selected dataset based upon the calculated similarity.

FIG. 4B depicts a user interface 420 for displaying results of a search of a search index, such as the search index 290, via a search application, such as the search application 272. The user interface 420 may be displayed by a personal electronic device, such as the personal electronic device 255, interfacing with a search application, such as the search application 273, hosted by a protein analysis platform, such the protein analysis platform 275. More particularly, the user interface 420 may be presented in response to a user interaction with the element 410 of the user interface 400.

As illustrated, the user interface 420 includes a results table configured to have a name column 422 and a vector similarity column 424. The vector similarity column 424 indicates the output of the similarity operation when comparing the embedding vector for the input protein sequence to the embedding vector for the protein indicated by the name column 422. In the illustrated example, the similarity calculation is normalized to scale from 0 to 1, but other embodiments may represent similarity in other manners. Additionally, while the user interface 420 only displays the top ten results (as determined by largest vector similarity), any number of the results may be viewable via navigation elements (not depicted) that enable a user to scroll or paginate through a list of results.

It should be appreciated that the user interfaces 400, 420 are merely one example of a user interface via which the disclosed functionality may be implemented. Alternate user interfaces may implement different types of user interface elements, including those adapted for different types of personal electronic devices. Additionally, in some embodiments, elements of the user interfaces 400, 420 may be divided across different panels, tabs, pop-outs, or other user interface constructs.

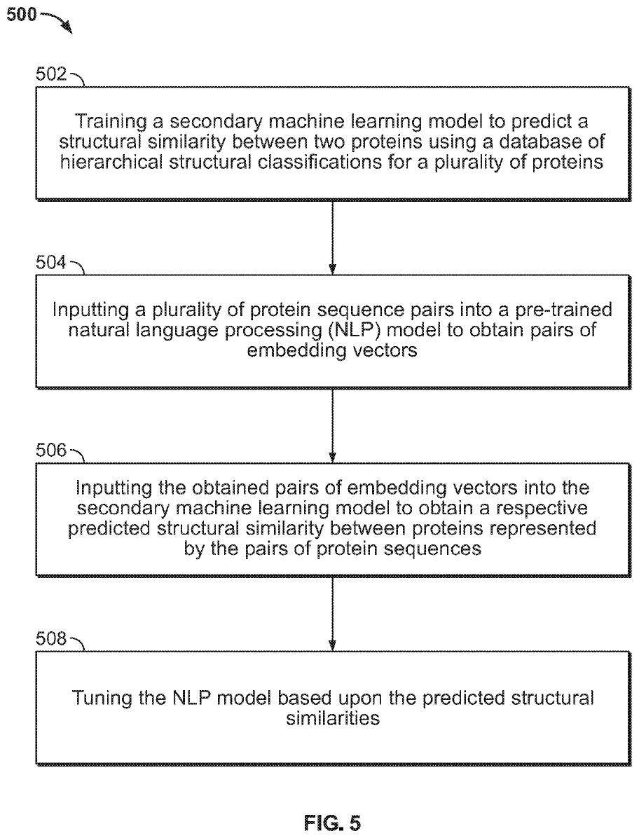

FIG. 5 depicts an example method 500 for training a model to predict functional similarity between protein sequences. More particularly, the method 500 fine-tunes a pre-trained natural language processing (NLP) model such that the output embedding vector indicates structural features of protein corresponding to an input protein sequence. The NLP model may include at least one of a Bayesian mixture model, a hidden Markov model, a dynamic Bayesian network model, a deep multilayer perceptron (MLP) model, a convolutional neural network (CNN) model, a recursive neural network (RvNN) model, recurrent neural network (RNN) model, a long short-term memory (LSTM) model, a sequence-to-sequence model, a neural transformer model, or a shallow neural network model. The method 500 may be performed by one or more processors of a protein analysis platform, such as the protein analysis platform 275, and/or a separate computing environment that interfaces with the protein analysis platform. In some embodiments, the method 500 is performed in accordance with a set of computer-readable instructions that form a training application, such as the training application 271.

The method 500 begins at block 502 when the one or more processors train a secondary machine learning model (such as the top model 125) to predict a structural similarity between two proteins using a database of hierarchical structural classifications (such as the database 282) for a plurality of proteins. In some embodiments, the database includes at least one of a Structural Classification of Proteins (SCOP), a Structural Classification of Proteins-extended (SCOPe) database, a Pfam database, or a proprietary hierarchical classification database.

To train the secondary machine learning model, the one or more processors may be configured to obtain, from the database of hierarchical structural classifications, the hierarchical structural classifications for a plurality of proteins. The one or more processors then obtain embedding vectors output by the NLP model for a pair of proteins from the plurality of proteins and input the embedding vectors (and, in some embodiments, the difference therebetween) into the secondary machine learning model to predict a structural similarity between the pair or proteins. Using the indications from the database of hierarchical structural classifications as a truth, the one or more processors re-train the secondary machine learning model based upon the predicted structural similarity. As a result, the secondary machine learning model is trained to predict a structural similarity metric that is based upon the hierarchical structural classifications indicated by the database.

At block 504, the one or more processors input a plurality of protein sequence pairs into the pre-trained NLP model to obtain pairs of embedding vectors. At block 506, the one or more processors input the obtained pairs of embedding vectors into the secondary machine learning model to obtain a respective predicted structural similarity between proteins represented by the pairs of protein sequences.

At block 508, the one or more processors tune the NLP model based upon the predicted structural similarities. In one embodiment, the one or more processors apply a target-based tuning technique that used that uses the predicted structural similarity as the target. For example, the tuning technique may apply gradient descent techniques. In some embodiments, the one or more processors continue tuning the NLP model until a training metric threshold (e.g., accuracy, test loss, validation loss, a combination thereof) is reached. In some embodiments, upon reaching the training metric threshold, the one or more processors store the tuned NLP model in a memory of the protein analysis platform.

In some embodiments, the one or more processors may be configured to perform the method 500 to tune a plurality of different pre-trained NLP models and/or to tune the pre-trained NLP models using a secondary model trained on a different set of hierarchical classification data. Additionally, after the one or more processors finish tuning the one or more NLP models, the one or more processors may build a search index that includes embedding vectors generated by the trained NLP models. Building the search index may be performed in accordance with a set of computer-readable instructions that form an indexing application, such as the indexing application 272.

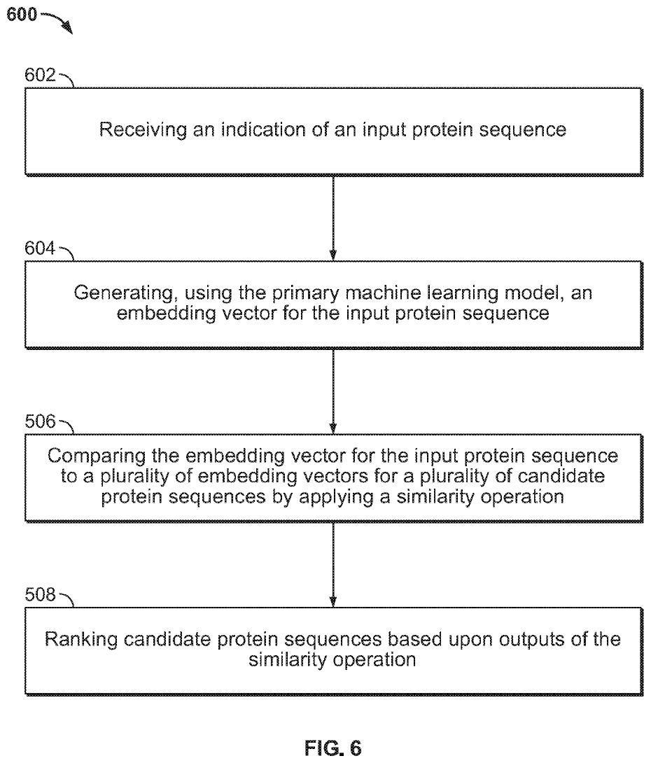

FIG. 6 depicts an example method 600 for predicting functional similarity between proteins using a primary machine learning model. In some embodiments, the primary machine learning model is a pre-trained NLP model that was tuned via the method 500. The method 600 may be performed by one or more processors of a protein analysis platform, such as the protein analysis platform 275. In some embodiments, the method 600 is performed in accordance with a set of computer-readable instructions that form a search application, such as the search application 273.

At block 602, the one or more processors receive an indication of an input protein sequence. For example, the protein sequences may be input into the text field 406 of the user interface 400.

At block 604, the one or more processors generate, using the primary machine learning model, an embedding vector for the input protein sequence. In some embodiments, the protein analysis platform may store a plurality of tuned NLP models. That is, the primary machine learning model is a first pre-trained NLP model fine-tuned via the secondary machine learning model and the one or more processors also interface with a second pre-trained NLP model fine-tuned via the secondary machine learning model. Similarly, the NLP models may be tuned using different secondary machine learning models. That is, the secondary machine learning model is a first secondary machine learning model trained using structural classifications maintained at a first structural classification database and the one or more processors also interface with a second pre-trained NLP model fine-tuned via a second secondary machine learning model trained using structural classifications maintained at a second structural classification database. In some of these embodiments, the one or more processors are configured to present a user interface via which a selection of either the first pre-trained NLP model or the second pre-trained NLP model is detected. For example, the user interface may include the selection element 402 to detect the selection of a specific NLP model. Accordingly, the one or more processors may generate the embedding vector for the input protein sequence using the selected NLP model.

At block 606, the one or more processors compare the embedding vector for the input protein sequence to a plurality of embedding vectors for a plurality of candidate protein sequences by applying a similarity operation. For example, the similarity operation may be one of a dot product, a cosine similarity, or a Euclidean similarity.

At block 608, the one or more processors rank the candidate protein sequences based upon outputs of the similarity operation. Generally, the higher the value of the similarity output, the more structurally similar the input protein sequence is to the candidate protein sequence. Accordingly, ranking the candidate protein sequences using the outputs of the similarity operation enables the detection of candidate protein sequence corresponding to proteins that are most likely to be structurally similar to the input protein. In some embodiments, the one or more processors presents a user interface that presents a listing of the ranked candidate proteins. For example, the one or more processors may be configured to generate the user interface 420. Because protein function is correlated to protein structure, the ranked list of structurally similar candidate proteins identifies candidate proteins likely to have a similar function (e.g., insecticide) to the input protein.

### Additional Considerations

This detailed description is to be construed as exemplary only and does not describe every possible embodiment, as describing every possible embodiment would be impractical, if not impossible. One may implement numerous alternate embodiments, using either current technology or technology developed after the filing date of this application.

Furthermore, although the present disclosure sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims. Although the following text sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated.

Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In exemplary embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software), may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of some of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

The patent claims at the end of this patent application are not intended to be construed under 35 U.S.C. § 112(f) unless traditional means-plus-function language is expressly recited, such as “means for” or “step for” language being explicitly recited in the claim(s).

This detailed description is to be construed as exemplary only and does not describe every possible embodiment, as describing every possible embodiment would be impractical, if not impossible. One may be implement numerous alternate embodiments, using either current technology or technology developed after the filing date of this application.

What is claimed is:

1. A system for predicting functional similarity between proteins, the system comprising:
one or more processors;
a first one or more non-transitory memories configured to store a primary machine learning model configured to convert a protein sequence for a protein into an embedding vector representative of features of the protein, wherein the primary machine learning model is trained or fine-tuned by:
training a secondary machine learning model to predict a structural similarity between two proteins,
inputting a plurality of protein sequence pairs into the primary machine learning model to obtain pairs of embedding vectors,
inputting the obtained pairs of embedding vectors into the secondary machine learning model to obtain a respective predicted structural similarity between proteins represented by the pairs of protein sequences, and
tuning the primary machine learning model based upon the predicted structural similarities,
a second one or more non-transitory memories configured to store processor-executable instructions that, when executed by the one or more processors, cause the system to:
receive an indication of an input protein sequence;
generate, using the primary machine learning model, an embedding vector for the input protein sequence;
compare the embedding vector for the input protein sequence to a plurality of embedding vectors for a plurality of candidate protein sequences by applying a similarity operation; and
ranking candidate protein sequences based upon outputs of the similarity operation for database search of functionally similar proteins.

2. The system of claim 1, wherein the primary machine learning model is a pre-trained natural language processing model.

3. The system of claim 1, wherein the primary machine learning model includes at least one of a Bayesian mixture model, a hidden Markov model, a dynamic Bayesian network model, a deep multilayer perceptron (MLP) model, a convolutional neural network (CNN) model, a recursive neural network (RvNN) model, recurrent neural network (RNN) model, a long short-term memory (LSTM) model, a sequence-to-sequence model, a neural transformer model, or a shallow neural network model.

4. The system of claim 1, wherein the primary machine learning model includes a normalizer configured to normalize a length of the output embedding vector.

5. The system of claim 1, wherein the secondary machine learning model is trained by:
obtaining, from a database, hierarchical structural classifications of a plurality of proteins,
obtaining embedding vectors output by the neural network model for a pair of proteins from the plurality of proteins,
inputting the embedding vectors into the secondary machine learning model to predict a similarity between the pair or proteins; and
re-training the secondary machine learning model using the hierarchical structural classifications for the pair of proteins as a truth.

6. The system of claim 5, wherein the database includes at least one of a Structural Classification of Proteins (SCOP), a Structural Classification of Proteins-extended (SCOPe) database, a Pfam database, or a proprietary hierarchical classification database.

7. The system of claim 1, wherein the similarity operation is one of a dot product, a cosine similarity, or a Euclidean similarity.

8. The system of claim 1, wherein:
the primary machine learning model is a first pre-trained NLP model fine-tuned via the secondary machine learning model; and
the first one or more non-transitory memories configured to store a second pre-trained NLP model fine-tuned via the secondary machine learning model.

9. The system of claim 8, wherein to generate the embedding vector for the input protein sequence, the instructions, when executed by the one or more processors, cause the system to:
present a user interface via which the system detects a selection of either the first pre-trained NLP model or the second pre-trained NLP model; and
generate, using the selected pre-trained NLP model, the embedding vector for the input protein sequence

10. The system of claim 1, wherein:
the secondary machine learning model is a first secondary machine learning model trained using structural classifications maintained at a first structural classification database; and
the first one or more non-transitory memories configured to store a second pre-trained NLP model fine-tuned via a second secondary machine learning model trained using structural classifications maintained at a second structural classification database.

11. A computer-implemented method for predicting functional similarity between proteins using a primary machine learning model configured to convert a protein sequence for a protein into an embedding vector representative of features of the protein, wherein the primary machine learning model is trained by (i) training a secondary machine learning model to predict a structural similarity between two proteins, (ii) inputting a plurality of protein sequence pairs into the primary machine learning model to obtain pairs of embedding vectors, (iii) inputting the obtained pairs of embedding vectors into the secondary machine learning model to obtain a respective predicted structural similarity between proteins represented by the pairs of protein sequences, and (iv) tuning the primary machine learning model based upon the predicted structural similarities, the method comprising:
receiving, via one or more processors, an indication of an input protein sequence;
generating, using the primary machine learning model, an embedding vector for the input protein sequence;
comparing, via the one or more processors, the embedding vector for the input protein sequence to a plurality of embedding vectors for a plurality of candidate protein sequences by applying a similarity operation; and
ranking, via the one or more processors, candidate protein sequences based upon outputs of the similarity operation for database search of functionally similar proteins.

12. The method of claim 11, wherein the primary machine learning model is a pre-trained natural language processing model.

13. The method of claim 12, wherein the primary machine learning model includes a normalizer configured to normalize a length of the output embedding vector.

14. The method of claim 11, wherein the secondary machine learning model is trained by:
obtaining, from a database, hierarchical structural classifications for a plurality of proteins,
obtaining embedding vectors output by the neural network model for a pair of proteins from the plurality of proteins,
inputting the embedding vectors into the secondary machine learning model to predict a structural similarity between the pair or proteins; and
re-training the secondary machine learning model using the hierarchical structural classifications for the pair of proteins as a truth.

15. The method of claim 14, wherein the database includes at least one of a Structural Classification of Proteins (SCOP), a Structural Classification of Proteins—extended (SCOPe) database, a Pfam database, or a proprietary hierarchical classification database.

16. The method of claim 11, wherein the similarity operation is one of a dot product, a cosine similarity, or a Euclidean similarity.

17. The method of claim 11, wherein:
the primary machine learning model is a first pre-trained NLP model fine-tuned via the secondary machine learning model; and
the method comprises interfacing, via the one or more processors, with a second pre-trained NLP model fine-tuned via the secondary machine learning model.

18. The method of claim 17, wherein generating the embedding vector for the input protein sequence comprises:
presenting, via the one or more processors, a user interface via a selection of either the first pre-trained NLP model or the second pre-trained NLP model is detected; and
generating, using the selected pre-trained NLP model, the embedding vector for the input protein sequence

19. The method of claim 11, wherein:
the secondary machine learning model is a first secondary machine learning model trained using structural classifications maintained at a first structural classification database; and
the method comprises interfacing, via the one or more processors, with a second pre-trained NLP model fine-tuned via a second secondary machine learning model trained using structural classifications maintained at a second structural classification database.

20. A computer-implemented method of training or fine-tuning a model to predict functional similarity between proteins comprising:
training, via one or more processors, a secondary machine learning model to predict a structural similarity between two proteins using a database of hierarchical structural classifications for a plurality of proteins;
inputting, via the one or more processors, a plurality of protein sequence pairs into a pre-trained natural language processing (NLP) model to obtain pairs of embedding vectors;
inputting, via the one or more processors, the obtained pairs of embedding vectors into the secondary machine learning model to obtain a respective predicted structural similarity between proteins represented by the pairs of protein sequences; and
tuning, via the one or more processors, the NLP model based upon the predicted structural similarities.
